(19) United States
(12) Plant Patent Application Publication
(10) Pub. No.: US 2008/0127378 P1
(43) Pub. Date: May 29, 2008

(54) Mandarin plant named Merbeingold 2336

(76) Inventor: Stephen Richard Sykes, Mildura (AU)

Correspondence Address: Gary J. Gershik;Cooper & Dunham LLP, 1185 Avenue of the Americas, New York, NY, 10036, US

(21) Appl. No.: 11/729,003

(22) Filed: Mar. 27, 2007

(30) Foreign Application Priority Data
Oct. 16, 2006 (AU) 2006/279 (PBR)

(51) Int. Cl. A01H 5/00

(52) U.S. Cl. PLT/202

(57) ABSTRACT

Merbeingold 2336 is a new Australian variety of mandarin. It was selected from the progeny obtained following a controlled pollination of Imperial mandarin×Ellendale tangor in 1984. It was selected because it is pollen sterile, strongly parthenocarpic and yields seedless fruits, which are sweet, juicy and easy-to-peel. Fruit maturity is early-to-mid season in the Murray Valley of Australia. Internally, the flesh of Merbeingold 2336 is attractive, orange in colour and tender with soft segment walls.

This application claims priority of Australian Plant Breeder's Right Application No. 2006/279, filed Oct. 16, 2006, the contents of which are incorporated by reference herein.

Merbeingold 2336 was accepted for Plant Breeder's Right (“PBR”) registration on Dec. 1, 2006 when a provisional PBR was granted. Acceptance was published in the Plant Varieties Journal (2007) Vol. 19, No 4, p23 (see, ipaustralia.gov.au/pdfs/plantbreed/PVJ_{—}19_{—}4.pdf). The Commonwealth of Australia Plant Breeder's Rights Act 1994 follows the guidelines of the International Convention for the Protection of New Varieties of Plants—UPOV Convention (1961), as revised at Geneva (1972, 1978 and 1991). Australia is a UPOV Member State.

### LATIN NAME OF THE GENUS AND SPECIES OF THE PLANT CLAIMED

Citrus reticulata×(C. reticulata×C. sinensis)

### VARIETY DENOMINATION

Merbeingold 2336

### BACKGROUND OF THE INVENTION

Merbeingold 2336 is a new variety of mandarin (Citrus species) bred by CSIRO Plant Industry. A copending application has been made for the variety Merbeingold 2350, which is a sibling selected from the same family as Merbeingold 2336.

Merbeingold 2336 is a mandarin variety selected from a family produced by making a controlled cross between Imperial mandarin (seed parent) with Ellendale tangor (pollen parent). Thus, the botanical name for the plant is:
Citrus reticulata×(C. reticulata×C. sinensis)

The plant may be used for horticultural production of mandarin fruits.

Merbeingold 2336 was selected from a family of 241 hybrids generated by a controlled cross-pollination of Imperial mandarin (maternal parent) with Ellendale tangor (pollen parent).

Imperial mandarin is an Australian variety that originated at Emu Plains, NSW, as a chance seedling in 1890. It is possibly a hybrid of the Mediterranean mandarin. Imperial trees are vigorous, upright and of medium size and they yield fruits that are early maturing.

Ellendale tangor is another Australian variety that was discovered as a chance seedling at Burrum, Queensland in 1878. Although its parentage is unknown, its characteristics and fruit size indicate that it is a tangor (mandarin×orange cross). Ellendale trees are generally large and of a spreading-round habit and produce large mid-to-late season fruit depending on where they are grown.

The seediness of fruits from both Imperial and Ellendale can be variable ranging from many-to-few-to-zero depending on the proximity of other sources of pollen. Both varieties are capable of producing fruits parthenocarpically.

CSIRO crossed Imperial with Ellendale to combine the characteristics of the two varieties and generate new parthenocarpic hybrids for selection of potential new varieties of seedless mandarins.

The controlled cross-pollination was conducted by emasculating an un-opened flower bud of the maternal parent and applying pollen from the paternal parent to the receptive stigma using a sterile soft-haired paintbrush. Pollen of Ellendale tangor was collected by drying anthers, which had been removed from unopened flower buds, in Petri dishes over silica gel in a dessicator. Dried dehisced anthers were stored in sealed glass vials over silica gel at 4° C. until needed. The cross was made in 1984 and the resultant seeds were extracted from fruits in 1985 and sown in a standard seed bed under glasshouse conditions. Emergent seedlings were transferred to a standard potting mix in pots and maintained under glasshouse conditions until they were rowed out in the breeding orchard at a planting density of 2 m within and 6 m between rows. Hybrid seedlings were maintained under irrigated orchard conditions thereafter. Standard citrus cultivation techniques were used to maintain the trees including application of fertilisers.

When hybrid 2336 flowered, it was subjected to a range of pollination treatments to assess its potential for producing seedless fruits. Fruits were harvested over 4 years and assessed for fruit quality, Based on the data collected, hybrid 2336 was selected for entry into second phase evaluation trials. The selection was entered into a comparative trial at CSIRO Plant Industry Koorlong (NW Victoria). It was also entered along with other selections into regional test plots with anonymous cooperating citrus growers under confidential testing agreement arrangements to protect inherent intellectual property. Based on its performance in these trials and test plots, hybrid 2336 was named Merbeingold 2336.

Daughter trees of Merbeingold 2336 propagated from the original seedling tree by asexual or vegetative means are uniform and stable. Similarly grand-daughter trees are uniform and stable. Trees of Merbeingold 2336 have been propagated by grafting or budding to seedling rootstocks, by top-working to established orchard trees and by rooting cuttings, confirming its uniformity and stability.

### BRIEF SUMMARY OF THE INVENTION

Fruits of Merbeingold 2336 are seedless even when challenged by viable pollen in an open-pollinated situation. The fruits are easy-to-peel and upon peeling, the segments are readily separated although some albedo tissue may adhere to the segments. Peeled segments are very tender with relatively soft walls meaning they are easy to consume and as a result the fruit has been described as melting. The juice has a °Brix of around 10 and an acid concentration of less than 1% at maturity, which gives a sweet flavour. The flavour and soft, easy-eating texture of the segments are favoured by children.

Though more orange than red-orange, the colour of the rind is more intense than Imperial mandarin. Like Imperial mandarin, rind strength is not high, which suggests the fruit may be more suited for domestic sales rather than shipping to more distant export markets.

Large fruits tend to be puffy, although cultural practices such as GA sprays have a positive effect to reduce this and improve rind quality. Fruits of Merbeingold 2336 can be snapped from the tree, but care should be taken as the button will be removed if harvesting is too violent. Fruits of Merbeingold 2336 are mature in the period June-through-July in Australia's Murray Valley, although the exact time for optimum quality will depend on rootstock and season. The range in fruit size tends to be similar to that of Imperial mandarin.

### BRIEF DESCRIPTION OF THE DRAWINGS

FIGS. 1A-1C: Show leaves from Merbeingold 2336 (FIG. 1A) and its parents, Imperial mandarin (FIG. 1B) and Ellendale tangor (FIG. 1C). In Merbeingold 2336 the Lamina length:width ratio=2.2±0.3; in Imperial mandarin the Lamina length:width ratio=2.7±0.2; and in Ellendale tangor the Lamina length:width ratio=2.1±0.2

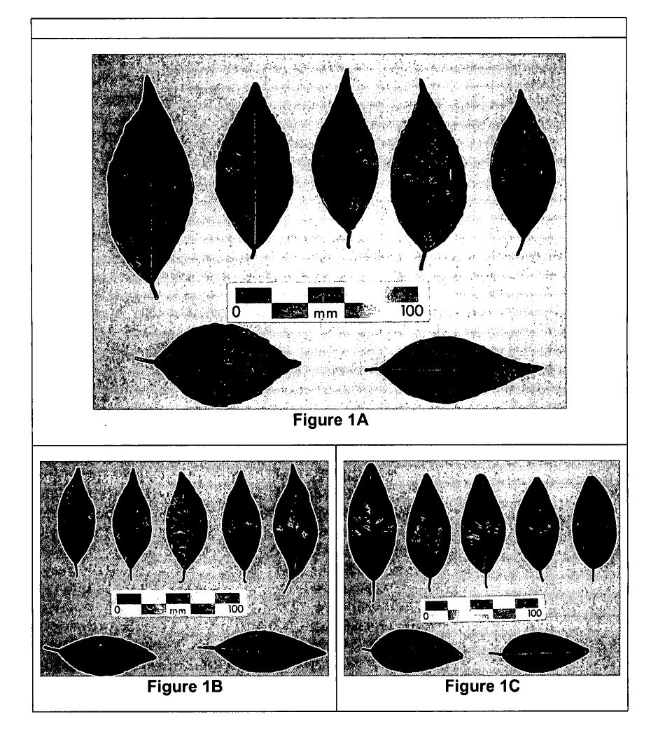

FIGS. 2A-2C: Show fruit of Merbeingold 2336 (FIG. 2A) and its parents, Imperial mandarin (FIG. 2B) and Ellendale tangor (FIG. 2C).

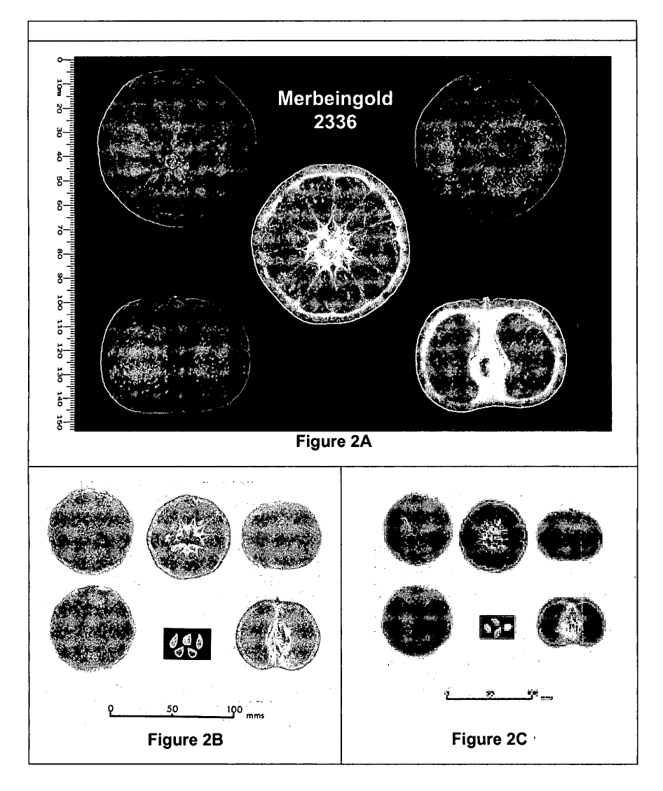

### DETAILED BOTANICAL DESCRIPTION

Variety—Merbeingold 2336
Citrus reticulata×(C. reticulata×C. sinensis)

Descriptors used herein from 1) European Union Community Plant Variety Office, Protocol for distinctness, uniformity and stability tests. Citrus L.—Group 1 MANDARINS. CPVO-TP\201\1 Adopted 18 Nov. 2004, and 2) IPGRI. (1999) Descriptors for Citrus. International Plant Genetic Resources Institute, Rome, Italy (ISBN 92-9043-425-2).
Plant: Diploid, growth habit spreading-to-upright, tree shape spheroid, density of branches medium, branch angle narrow, trunk surface smooth, shoot tip green and surface glabrous.
Spine (thorn): Absent on adult tree.
Leaf: Evergreen, type simple, shape ovate, intensity of green colour on lamina medium-to-dark, colour variegation absent, margin weakly dentate, shape of apex acute, length medium (91±11 mm), lamina width broad (42±6 mm), lamina length to width ratio 2.2±0.3, cross-sectional shape intermediate, lamina undulation slight, lamina attachment brevipetiolate, petiole length short (10±3), petiole wings obdeltate and very narrow, petiole attachment to stem curved, junction between petiole and lamina articulate, colour of upper/lower surface of lamina same.
Flower: Hermaphrodite, arrangement solitary and as a raceme, position axillary and terminal, length of petal medium, anther colour pale yellow-to-white, viable pollen absent, length of anther relative to style shorter (inferior), colour of open flower white, 5 petals per flower, stamens >4 per petal, style straight and complete, self-pollen reaction sterile.
Fruit: Maturity early-to-mid-season (June Australia), borne both inside and outside canopy, obloid, attachment to stalk medium-to-strong, broadest at equator, shape in transverse section circular, base truncate, apex depressed slightly, neck absent, slight depression at stalk end, number of radial grooves at stalk end intermediate, collar absent, distal part slightly concave-to-flattened, depression at distal end, areola absent, stylar end closed, stylar scar small, style not persistent, navel opening absent, radial grooves at distal end absent, rind surface orange, surface glossiness strong, rind surface smooth-to-very slightly pitted, oil glands all more-or-less same size, slight pitting of oil glands present pebbling absent, oil glands very weakly conspicuous, small and of low density, rind thickness medium (3-5 mm), rind adherence to flesh weak, rind strength medium, rind oiliness dry, albedo white and loose, amount of albedo adhering to flesh small-to-medium, albedo strands present, flesh medium orange, pulp colour uniform, pulp firmness soft, pulp texture fleshy, core medium, round and sparsely filled, segments uniform, rudimentary segments absent, number of well developed segments medium (7-12; mean 9.5±1.7), adherence of segment walls to each other medium, segment walls thin and weak, vesicle length medium, vesicle thickness medium, navel absent, juiciness medium-to-high, total soluble solids medium 9-11 °Brix, juice acidity low-to-medium, strength of fibre weak, parthenocarpy present.
Seed: Number zero under controlled manual self pollination, or 0-3 (mostly 0) under open-pollination dependent on proximity of pollen sources, monoembryonic, shape ovoid, length short, width narrow, surface smooth, external colour whitish/cream, colour of inner seed coat light brown, chalazal cream, cotyledons white-to-light yellow-cream.

What is claimed is:

1. A new and distinct pollen sterile, parthenocarpic mandarin variety named Merbeingold 2336 as illustrated and described.
